(19) United States
(12) Patent Application Publication
(10) Pub. No.: US 2011/0118551 A1
(43) Pub. Date: May 19, 2011

(54) COLLATERAL SOFT TISSUE PROTECTION SURGICAL DEVICE

(75) Inventors: Jeremy CIPOREN, Sacramento, CA (US); Jesse A. Dosher, Shoreline, WA (US); Diana C.W. Friedman, Seattle, WA (US); Blake Hannaford, Seattle, WA (US); Louis Kim, Seattle, WA (US); Kristen S. Moe, Seattle, WA (US); James S. Pridgeon, Seattle, WA (US); Dinesh Ramanathan, Seattle, WA (US); Laligam Sekhar, Seattle, WA (US)

(73) Assignee: SPI Surgical, Inc., Seattle, WA (US)

(21) Appl. No.: 12/943,779

(22) Filed: Nov. 10, 2010

Related U.S. Application Data
(60) Provisional application No. 61/261,310, filed on Nov. 14, 2009.
(60) Provisional application No. 61/293,932, filed on Jan. 11, 2010.
(60) Provisional application No. 61/346,476, filed on May 20, 2010.

(51) Int. Cl. A61B 1/32

(52) U.S. Cl. 600/201

(57) ABSTRACT

A collateral soft tissue protection surgical device protects collateral soft tissue from damage during a surgical procedure within a surgical space of a body. The device comprises an elongated flexible sheath having a proximal end and a distal end. The proximal end has a first opening and the distal end has a second opening. The sheath further comprises a side wall between the proximal and distal ends that defines the first and second openings. The side wall is conformal to the surgical space and arranged to resist perforation by surgical instruments in use during the surgical procedure, and also to define and maintain the access pathway to the surgical site.

### PRIORITY CLAIM

The present application claims the benefit of copending U.S. Provisional Patent Application Ser. No. 61/261,310, filed Nov. 14, 2009; the present application also claims the benefit of copending U.S. Provisional Application Ser. No. 61/293,932, filed Jan. 11, 2010; the present application also claims the benefit of copending U.S. Provisional Application Ser. No. 61/346,476, filed May 20, 2010; all of the foregoing applications are incorporated herein by reference in their entireties.

### BACKGROUND OF THE INVENTION

The present invention is directed to a surgical device. The present invention is more particularly directed to a surgical device that protects soft tissue from collateral damage during surgery.

Endoscopic surgery targeting lesions of the pituitary fossa, skull base, and nasopharynx are commonplace procedures in neurological surgery and otolaryngology. These are typically performed using a transnasal or sublabial route, but also can be carried out using a small eye-lid crease or conjunctival incision for a transorbital route. There are several advantages to endoscopic surgery of the brain, skull base and nasopharynx. It obviates large cranial incisions and bony openings, which require much more extensive exposures, brain retraction and wound healing. It provides optimal illumination and visualization of the target tissues because the camera of the endoscope is brought directly to the area of interest. Endoscopic surgery also permits target tissue treatment through small exposures and minimal bony openings to the skull.

However, in order to access the skull base and nasopharynx endoscopically, some local trauma is imparted to the nasal mucosa, turbinates, nasal septum, and sphenoid/frontal/maxillary sinus, and, in the case of transorbital approaches, orbital and periorbital tissue. This surgical pathway trauma can add to the trauma of the procedure and prolong the patient's recovery time. In addition, there is frequent and persistent “run down” of mucous, blood, and soiled irrigation fluid that obscures the endoscopic visualization. This leads to the constant need for irrigation and suction of the offending liquids, as well as the outright removal, cleaning and replacement of the endoscope. This can occur dozens of times during a single procedure, making the cleaning and clearing of the endoscope both time consuming and frustrating to the surgeon. Therefore, a device that can reduce or eliminate these aspects of endoscope surgery will reduce soft tissue trauma, shorten operative times, and potentially lead to improved patient outcomes.

Accessing the surgical site through any route, but especially through either a transnasal or transorbital route, may require the surgeon to travel around or through structures, which can be extremely time consuming. For more complex procedures, an additional surgeon is sometimes called in specifically to access the surgical site. Whenever an instrument needs to be substituted, or an endoscope needs to be cleaned, the critical structures are again put at risk as the devices are removed and reinserted. A device that can be inserted once and remain in place to define and maintain the access pathway, while allowing other devices to pass through it, would shorten operative times, reduce the risk of trauma to critical structures, and potentially lead to improved patient outcomes.

### SUMMARY

According to one embodiment, a collateral soft tissue protection surgical device protects collateral soft tissue from damage during a surgical procedure within a surgical space of a body. The device comprises an elongated flexible sheath having a proximal end and a distal end. The proximal end has a first opening and the distal end has a second opening. The sheath further comprises a side wall between the proximal and distal ends that defines the first and second openings. The side wall is conformal to the surgical space and arranged to resist perforation by surgical instruments in use during the surgical procedure, and also to define and maintain the access pathway to the surgical site.

The sheath may be expandable from a low profile shape to permit the device to be introduced into the surgical space, to an expanded shape to conform to the surgical space after being introduced into the surgical space. The sheath may be hour glass shaped.

Alternatively, the cross sectional dimension of the sheath at the proximal end may be less than the cross sectional dimension of the sheath at the distal end. For example, the sheath may be horn shaped at its proximal end and have a tapered shape that leads to a cylindrical shape at its distal end.

Alternatively, the cross sectional dimension of the sheath at the proximal end may be greater than the cross sectional dimension of the sheath at the distal end. For example, the proximal end of the sheath may be horn shaped.

The sheath may have an inner surface that is a low friction surface. The inner surface of the sheath may include a low friction coating. The outer surface of the sheath may be textured to provide gentle friction with the collateral soft tissue to assist in maintaining the sheath in place.

The sheath may include at least one irrigation channel. The sheath may alternatively or in addition include at least one suction channel.

The sheath side wall may include at least one cut-out to permit a collateral tissue projection to pass there through. Alternatively or in addition, the sheath may include at least one portion having radio-opaque material Still further, the sheath may include at least one portion having magnetic material.

The device may further comprise a light source for illuminating at least a portion of the surgical space. The light source may include an optical fiber. The optical fiber may be carried on the sheath.

According to another embodiment, a collateral soft tissue protection surgical device that protects collateral soft tissue from damage during a surgical procedure within a surgical space of a body comprises an elongated flexible sheath having a proximal end and a distal end. The proximal end has a first opening and the distal end has a second opening. The sheath further comprises a side wall between the proximal and distal ends that defines the first and second openings. The side wall is conformal to the surgical space and arranged to resist perforation by surgical instruments in use during the surgical procedure. The device further includes an irrigation system including at least one irrigation channel formed in the sheath and a light source for illuminating at least a portion of the surgical space. The light source includes at least one optical fiber extending down the sheath.

### BRIEF DESCRIPTION OF THE DRAWINGS

The features of the present invention which are believed to be novel are set forth with particularity in the appended claims. The invention, together with further features and advantages thereof, may best be understood by making reference to the following description taken in conjunction with the accompanying drawings, in the several figures of which like reference numerals identify identical elements, and wherein:

FIG. 1 is sectional view of a human head and surgical device embodying the present invention deployed in a nostril of the human head;

FIG. 2 is a sectional view, similar to FIG. 1 illustrating a condition of the device when first introduced into the nostril of the human head;

FIG. 3 is a cross-sectional view taken along lines 3-3 of FIG. 1, illustrating a pair of devices embodying the invention within respective nostrils;

FIG. 4 is sectional view of a human head and another surgical device embodying the present invention deployed in a nostril of the human head;

FIG. 5 is sectional view of a human head and still another surgical device embodying the present invention deployed in a nostril of the human head;

FIG. 6 is a perspective view of another device embodying the invention illustrating texturing on the outer surface thereof to facilitate retention of the device in the body;

FIG. 7 is a sectional side view of another device embodying the invention having an irrigation system therein;

FIG. 8 is a sectional side view of another device embodying the invention having a suction system therein;

FIG. 9 is a sectional side view of another device embodying the invention having perforated cutouts in the sidewall thereof;

FIG. 10 is a sectional side view of another device embodying the invention having radio opaque material within the sidewall thereof;

FIG. 11 is a sectional view similar to that of FIG. 3 illustrating a pair of devices embodying the invention within respective nostrils and each including magnets for retaining the devices in place during a surgical procedure;

FIG. 12 is a sectional side view of another device embodying the invention having optical fibers within the sidewall thereof;

FIG. 13 is a sectional side view of another device embodying the invention having optical fibers and an irrigation system within the sidewall thereof;

FIG. 14 is a side view, partly in section, illustrating another device embodying the invention being deployed; and

FIG. 15 is a side view, partly in section, illustrating the device of FIG. 14 after deployment.

### DETAILED DESCRIPTION

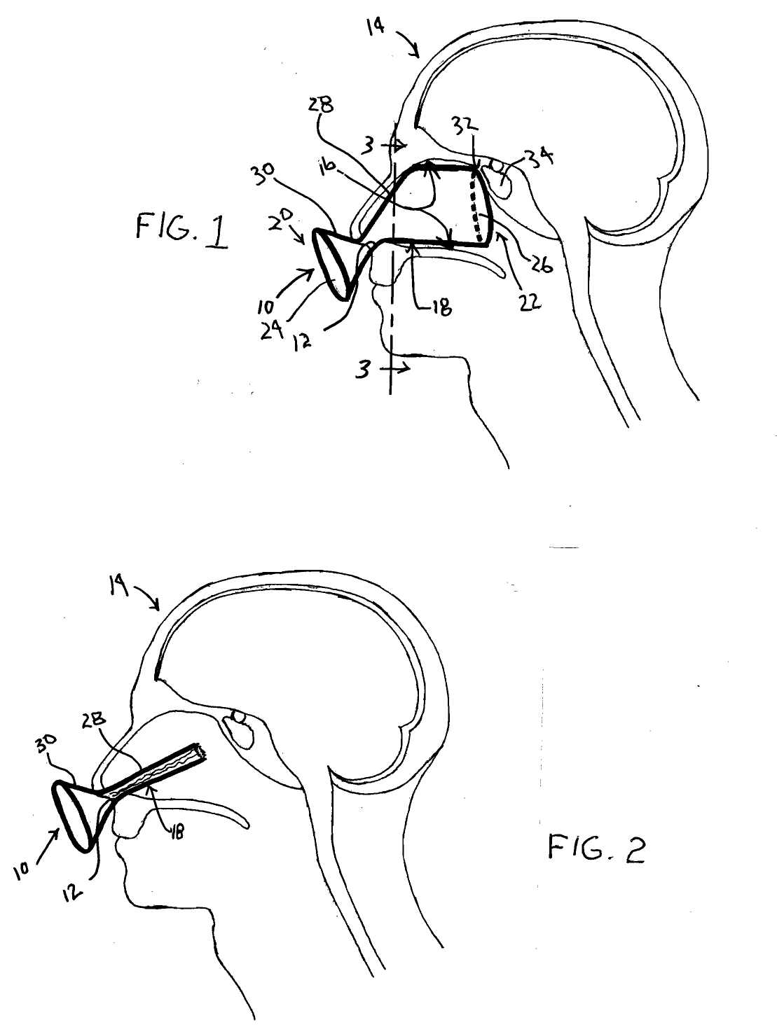

FIG. 1 shows a surgical device 10 embodying the present invention deployed within a nostril 12 of a human head 14. The device, as will be seen subsequently, is a collateral soft tissue protection surgical device that protects collateral soft tissue from damage during a surgical procedure within a surgical space 16 of a body. The device generally includes an elongated flexible sheath 18 having a proximal end 20 and a distal end 22. The proximal end has as a first opening 24 and the distal end has a second opening 26. A sidewall 28 between the proximal end 20 and the distal end 22 defines the openings 24 and 26. The sidewall 28 is conformal to the surgical space 16 and arranged to resist perforation by surgical instruments in use during the surgical procedure, and also to define and maintain the access pathway to the surgical site. The sheath may be formed of, for example, latex rubber, silicone rubber, latex or polymeric silicone substances, or other flexible polymer materials and/or other biocompatible elastic material.

The device 10 has a horn shaped portion 30 at its proximal end 20 that extends proximally from the nostril 12. The horn shape 30 together with the tapered side wall 28 serve to maintain the device in place during the surgical procedure. The horn shape 30 also permits instruments to be deployed through a wide angle range to fully address the surgical site 32. The surgical site may, for example, include a lesion 34 in need of removal.

FIG. 2 shows how the device 10 may be deployed in the nostril 12 of the patient's head 14. However, the devices of the present invention may be used to advantages in other surgical approaches as well including transorbital approaches or conjunctival incisions. Here it may be seen that the sidewall 28 of the sheath 18 is in a collapsed state distally from the horn shaped portion 30. This enables ready insertion of the sheath 18 into the nostril 12. Once the sheath 18 is positioned within the nostril 12 as illustrated, the sheath 18 may be expanded to conform to the surgical space. To that end, the sheath may have a natural full shape to which it naturally expands from a compressed condition once it is released. When expanded from the low profile shape as seen in FIG. 2 to the expanded shape, the sheath 18 will conform to the surgical space. This may be seen in the sectional view of FIG. 3. Here, two identical devices 10 are deployed on either side of the septum 36. The sidewalls 28 of the device 10 are fully conformed to the inner wall of the nostrils 12. By conforming to the inner wall of the nostrils 12, the devices 10 also define and maintain the access pathway to the surgical site. In addition, each device 10 has an inner surface 25. The inner surface 25 may be coated with a low friction coating. Suitable coating materials include, for example, PTFE, hyaluronan, and glycerin. This makes the inner surface a low friction surface to assist in easier insertion of instruments into the sheath 18 and avoiding piercing the device and the collateral soft tissue with the instruments.

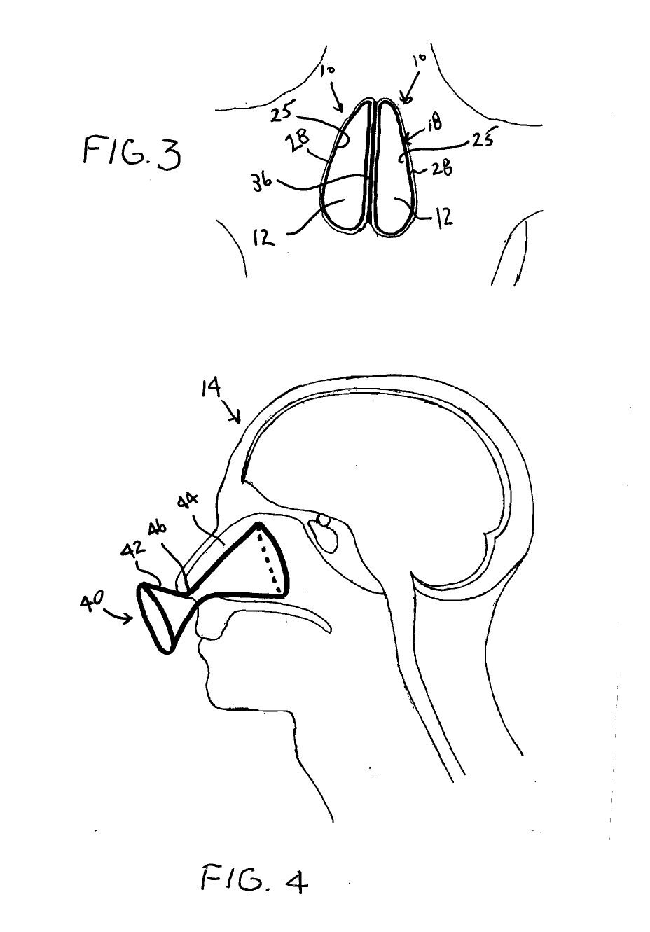

FIG. 4 illustrates another device 40 embodying the invention. Here, the device 40 is hour glass shaped having a flared proximal portion 42 and a flared distal portion 44. A reduced dimension section 46 joins the portions 42 and 44 and serves to hold the device 40 in place during the surgery. The device 40 may be deployed in the same manner as previously described with respect to the device 10 of FIG. 1.

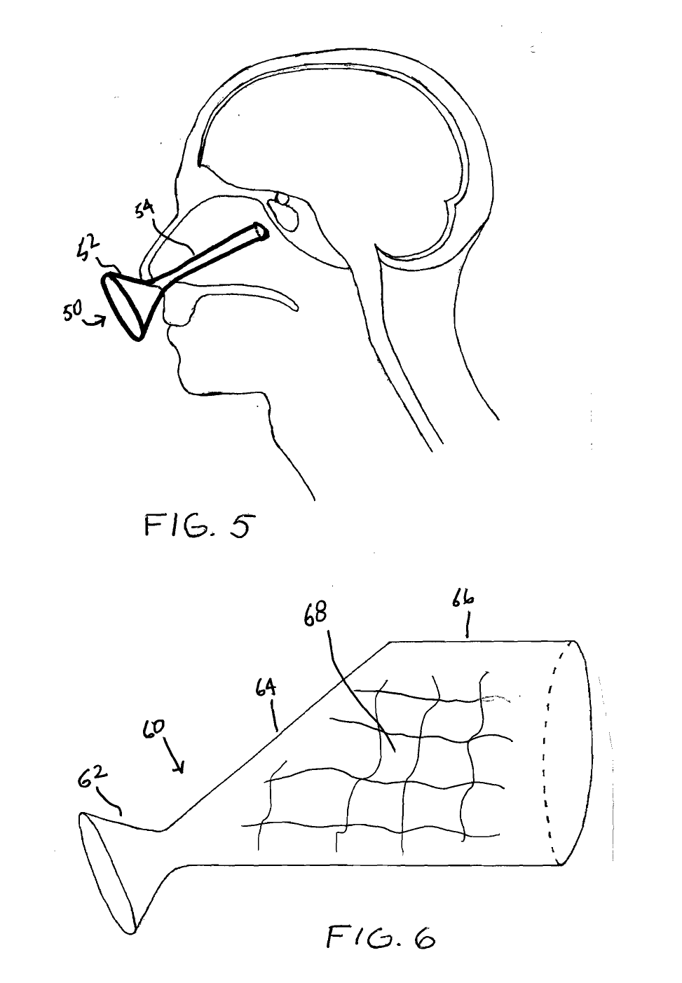

FIG. 5 illustrates another device 50 embodying the invention. Here, the device 50 is horn shaped in a proximal portion 52 and elongated in distal portion 54. This device may be used to advantage when the surgical target of relatively small size, not requiring surgical instruments to be deployed through a wide angle range to fully address the surgical site.

FIG. 6 shows another device 60 embodying further aspects of the invention. The device 60 is shaped similarly to the device 10 of FIG. 1 and has a horn shape 62 at its proximal end and a tapered shape 64 that leads to a cylindrical shape 66 at its distal end. The device 60 may be formed from any of the materials previously mentioned. The device 60 further has a textured sidewall 68. The textured sidewall 68 provides a gentle friction with the collateral soft tissue to assist in maintaining the sheath in place. The texturing may be included in devices of any shape including the horn or hourglass configurations disclosed herein.

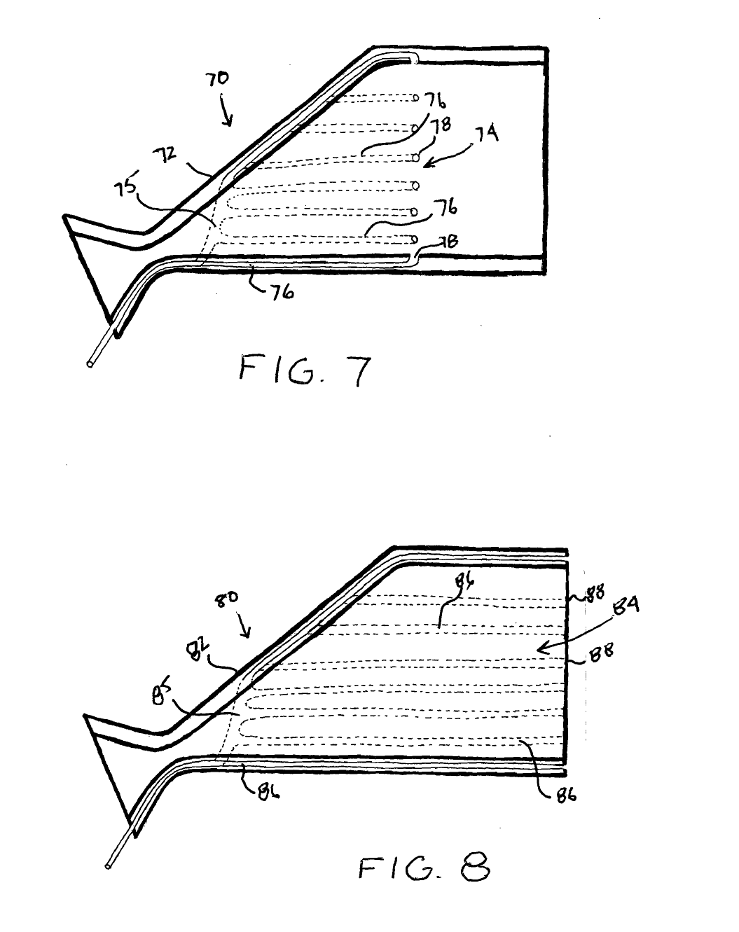

FIG. 7 illustrates another device 70 embodying further aspects of the invention. The device 70 is shaped like the device 10 of FIG. 1 and can be formed from the same materials previously mentioned. Here however, the device includes an irrigation system 74 within its sidewall 72. More specifically, the sidewall has an internal feed channel 75 that communicates with internal distribution channels 76. The distribution channels terminate at ports 78 to admit cleaning solution, such as saline solution, for example, into the surgical site. As a result, the surgical site may be cleaned without the need for the removal of surgical instruments, such as an endoscope, from the surgical site. As may be appreciated, the irrigation system could also be included in devices having the horn or hourglass configurations as well.

FIG. 8 illustrates another device 80 embodying further aspects of the invention. The device 80 is also shaped like the device 10 of FIG. 1 and can be formed from the same materials previously mentioned. Here however, the device includes a suction system 84 within its sidewall 82. More specifically, the sidewall has an internal common channel 85 that communicates with internal branch channels 86. The branch channels extend all of the way to the end of the device 80 and terminate at ports 88. The extended branch channels 86 render the device capable of providing suction for removal of fluids such as “run down” of blood, mucous, and soiled irrigation fluid that may obscure endoscopic visualization. Of course, the suction system could also be present on the devices having the horn or hourglass configurations as well.

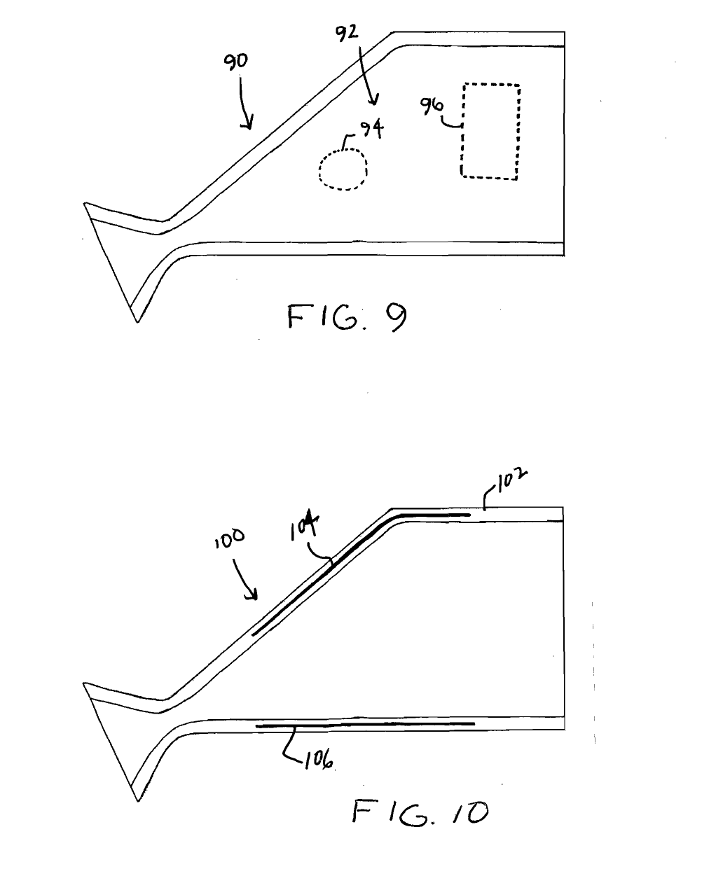

FIG. 9 illustrates another device 90 embodying further aspects of the invention. The device 90 is also shaped like the device 10 of FIG. 1 and can be formed from the same materials previously mentioned. Here however, the device includes perforated cutouts 94 and 96 within the sidewall 92 of the device 90. The cutouts assist in removing portions of the sidewall 92 should it be necessary to permit collateral projecting tissue to extend there through. This not only facilitates retention of the device, but also potential removal of the projecting tissue should that be necessary. As may be appreciated, the cutouts could also be present on any of the disclosed embodiments herein including the devices having the horn or hourglass configurations.

FIG. 10 illustrates another device 100 embodying further aspects of the invention. The device 100 is also shaped like the device 10 of FIG. 1 and can be formed from the same materials previously mentioned. Here however, the device includes radio opaque material 104 and 106 within the sidewall 102 of the device 100. Since the radio opaque material 104 and 106 is within the sidewall 102 of the device 100, and since the sidewall conforms to the shape of the surgical space, the margins of the surgical space will be clearly visible under fluoroscopy during a surgical procedure. The radio opaque material will also make the presence of the device 100 obvious under fluoroscopy to assist in guarding against the potential for the device 100 to be left in the patient after the surgical procedure is completed. The radio opaque material could also incorporated into any of the devices disclosed herein, including the devices having the horn or hourglass configurations.

FIG. 11 is a sectional view similar to the sectional view of FIG. 3. Here it may be seen that a pair of devices, devices 110 and 116 have been deployed on opposite sides of a septum 36. Device 110 has sidewall 112 and device 116 has sidewall 117. Sidewall 112 carries magnets 113 and 114 and sidewall 117 carries magnets 118 and 119. The magnets are positioned so that magnet 113 is opposite magnet 118, and magnet 114 is opposite magnet 119. The attraction between the magnet pairs serves to gently hold the device 110 and 116 in place during the surgical procedure employing the devices 110 and 116.

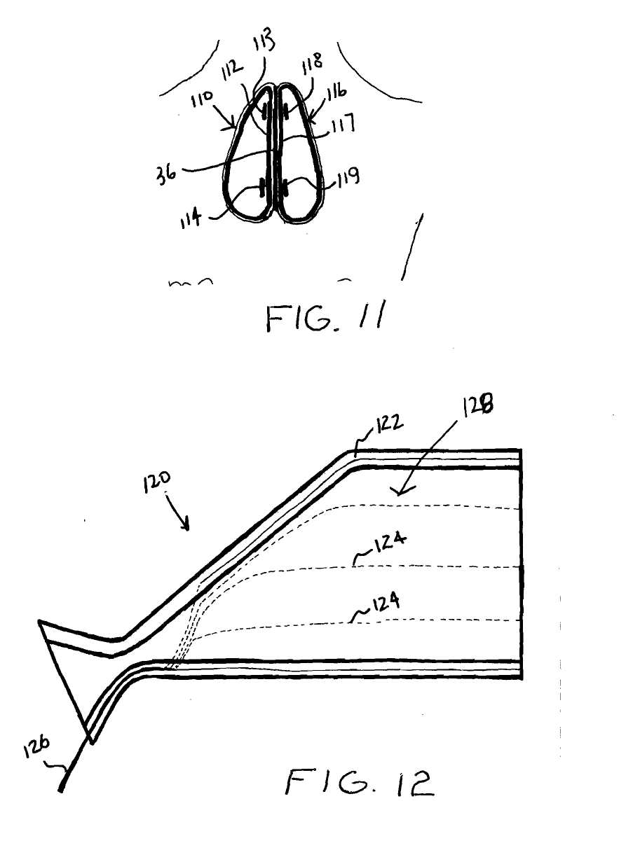

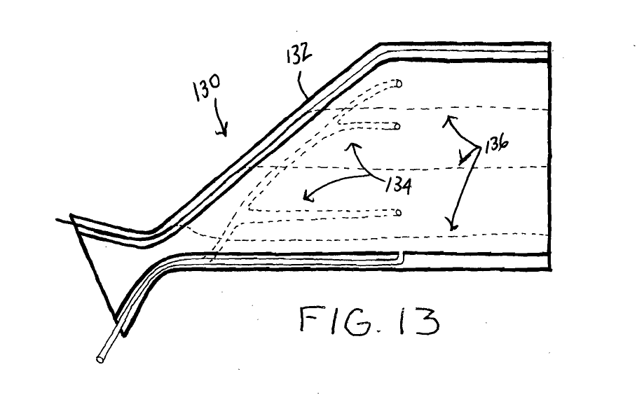

FIG. 12 illustrates another device 120 embodying further aspects of the invention. The device 120 is also shaped like the device 10 of FIG. 1 and can be formed from the same materials previously mentioned. Here however, the device includes a light projection system 128 within its sidewall 122. More specifically, the sidewall 122 has an internal common optical fiber 126 that serves as a light source and is coupled to internal branch optical fibers 124. The branch optical fibers 124 extend all of the way to the end of the device 120. The extended optical fibers 124 render the device capable of projecting light from the end of the device 120 onto the surgical site. This supports visualization of the surgical procedure. Light for the common source 126 may be obtained from a light emitting diode or other source known in the art. As may be appreciated, the light projection system could also be employed in any of the devices disclosed herein, including the devices having the horn or hourglass configurations.

FIG. 13 illustrates another device 130 embodying further aspects of the invention. The device 130 is also shaped like the device 10 of FIG. 1 and can be formed from the same materials previously mentioned. Here however, the device includes a combination irrigation system 134 and light projection system 136. The irrigation system 134 and light projection system 136 are formed in the sidewall 132 in the same manner as previously described. The combination irrigation system and light projection system may also be included in any of the devices disclosed herein, including the device having the horn or hourglass configurations.

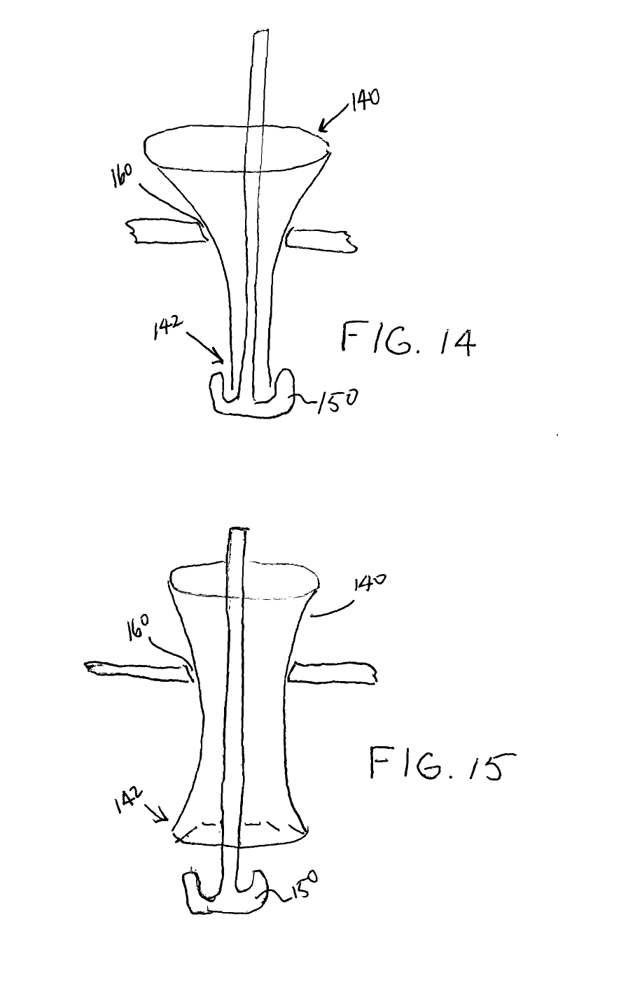

Referring now to FIGS. 14 and 15, they show another method of deploying a device 140 embodying the invention. The device 140 has an hour glass configuration and may be formed from any of the materials previously described. In FIG. 14, it may be seen that an end 142 of the device 140 has been gathered and collapsed into a deployment tool 150. As will be noted in FIG. 14, the deployment tool 150 is inserted into an opening 160 formed within the skull, for example. When the device 140 is in place, the device 140 is held stationary while the tool 150 is moved out of the device 140. This allows the end 142 of the device 140 to expand to its full configuration. The tool 150 may now be removed through the device 140 to complete the device deployment.

From the foregoing, it can be seen that the invention provides surgical devices that protect collateral soft tissue from damage during a surgical procedures, and also define and maintain the access pathway to the surgical site. The devices may incorporate many different functions to assist in the surgery including irrigation, suction, and light projection. The devices are shaped to afford wide angle instrument use to address large surgical sites. By virtue of the present invention, soft tissue trauma is reduced, operating times are reduced, and improved patient outcomes are made possible.

While particular embodiments of the invention has been shown and described, changes and modifications may be made. It is therefore intended to cover in the appended claims all such changes and modifications which fall within the true spirit and scope of the invention.

What is claimed is:

1. A collateral soft tissue protection surgical device that protects collateral soft tissue from damage during a surgical procedure within a surgical space of a body, the device comprising an elongated flexible sheath having a proximal end and a distal end, the proximal end having a first opening and the distal end having a second opening, the sheath further comprising a side wall between the proximal and distal ends that defines the first and second openings, the side wall being conformal to the surgical space and arranged to resist perforation by surgical instruments in use during the surgical procedure.

2. The device of claim 1, wherein the sheath is expandable from a low profile shape to permit the device to be introduced into the surgical space, to an expanded shape to conform to the surgical space after being introduced into the surgical space.

3. The device of claim 1, wherein the sheath is hour glass shaped.

4. The device of claim 1, wherein the cross sectional dimension of the sheath at the proximal end is less than the cross sectional dimension of the sheath at the distal end.

5. The device of claim 4, wherein the sheath may be horn shaped at its proximal end and have a tapered shape that leads to a cylindrical shape at its distal end.

6. The device of claim 1, wherein the cross sectional dimension of the sheath at the proximal end is greater than the cross sectional dimension of the sheath at the distal end.

7. The device of claim 6, wherein the proximal end of the sheath is horn shaped.

8. The device of claim 1, wherein the sheath has an inner surface, and wherein the inner surface is a low friction surface.

9. The device of claim 8, wherein the inner surface of the sheath includes a low friction coating.

10. The device of claim 1, wherein the sheath has an outer surface, and wherein the outer surface is textured to provide gentle friction with the collateral soft tissue to assist in maintaining the sheath in place.

11. The device of claim 1, wherein the sheath includes at least one irrigation channel.

12. The device of claim 1, wherein the sheath includes at least one suction channel.

13. The device of claim 1, wherein the sheath side wall includes at least one cut-out to permit a collateral tissue projection to pass there through.

14. The device of claim 1, wherein the sheath includes at least one portion having radio-opaque material

15. The device of claim 1, wherein the sheath includes at least one portion having magnetic material.

16. The device of claim 1, further comprising a light source for illuminating at least a portion of the surgical space.

17. The device of claim 16, wherein the light source includes an optical fiber.

18. The device of claim 17, wherein the optical fiber is carried on the sheath.

19. A collateral soft tissue protection surgical device that protects collateral soft tissue from damage during a surgical procedure within a surgical space of a body, the device comprising:
an elongated flexible sheath having a proximal end and a distal end, the proximal end having a first opening and the distal end having a second opening, the sheath further comprising a side wall between the proximal and distal ends that defines the first and second openings, the side wall being conformal to the surgical space and arranged to resist perforation by surgical instruments in use during the surgical procedure;
an irrigation system including at least one irrigation channel formed in the sheath; and
a light source for illuminating at least a portion of the surgical space, the light source including at least one optical fiber extending down the sheath.
